(19) United States
(12) Patent Application Publication
(10) Pub. No.: US 2016/0058619 A1
(43) Pub. Date: Mar. 3, 2016

(54) EAR PROTECTOR AGAINST MOISTURE

(71) Applicant: OIDO NOVA LTD., Qiryat Motzkin (IL)

(72) Inventors: Liat GOLDFARB, Qiryat Motzkin (IL); David Natan ARLINSKY, Atlit (IL)

(21) Appl. No.: 14/471,009

(22) Filed: Aug. 28, 2014

(51) Int. Cl. A61F 11/10

(52) U.S. Cl. CPC A61F 11/10

(57) ABSTRACT

An ear protector device includes an earplug to insert into an ear canal of a user. The earplug includes a plurality of inflatable structures arranged along a longitudinal axis of the earplug. A length of the earplug is extendible to increase a distance between two of the inflatable structures. Each inflatable element is laterally inflatable when inserted into the ear canal so as to substantially fill a cross section of the ear canal.

### FIELD OF THE INVENTION

The present invention relates to an ear protector to prevent penetration of moisture into an ear canal.

### BACKGROUND OF THE INVENTION

In the case of some ear conditions or disorders, care must be taken to prevent liquids or moisture from penetrating into the ear canal (external auditory meatus). In particular, liquids must be prevented from coming into contact with the ear canal and/or eardrum (tympanic membrane). Such disorders include, for example, external otitis or a perforated eardrum.

The need for preventing the entry of water into the ear canal may severely limit the activities of a person who is suffering from such an ear disorder. For example, the person may be required to avoid swimming other water-based sport, recreation, or activities. Such a person may be prevented from showering or bathing in the usual manner.

### SUMMARY OF THE INVENTION

There is thus provided, in accordance with some embodiments of the present invention, an ear protector device comprising an earplug to insert into an ear canal of a user, the earplug including a plurality of inflatable structures arranged along a longitudinal axis of the earplug, a length of the earplug being extendible to increase a distance between two of the inflatable structures, each inflatable element being laterally inflatable when inserted into the ear canal so as to substantially fill a cross section of the ear canal.

Furthermore, in accordance with some embodiments of the present invention, the device includes a longitudinally inflatable channel that is inflatable to lengthen the earplug.

Furthermore, in accordance with some embodiments of the present invention, the device includes a pump for inflating the channel.

Furthermore, in accordance with some embodiments of the present invention, each structure of the plurality of inflatable structures is arranged axially symmetrically about the earplug.

Furthermore, in accordance with some embodiments of the present invention, each structure of the plurality of inflatable structures is connected to a common longitudinal channel by a radial channel, the structure being inflatable via that radial channel.

Furthermore, in accordance with some embodiments of the present invention, the device includes a widened base.

Furthermore, in accordance with some embodiments of the present invention, the device includes a pump for inflation of the inflatable structures.

Furthermore, in accordance with some embodiments of the present invention, the device includes at least one earmuff for covering an outer ear of the user.

Furthermore, in accordance with some embodiments of the present invention, an outer surface of the earmuff is impermeable to moisture.

Furthermore, in accordance with some embodiments of the present invention, the earmuff includes a resilient rim for placement against a head of the user to form a seal against moisture.

Furthermore, in accordance with some embodiments of the present invention, the earmuff includes a cartridge containing a hygroscopic material.

Furthermore, in accordance with some embodiments of the present invention, the device includes a pump to cause air from within the earmuff to flow through the hygroscopic material.

Furthermore, in accordance with some embodiments of the present invention, the at least one earmuff includes two earmuffs attached to a rigid bow.

Furthermore, in accordance with some embodiments of the present invention, the at least one earmuff includes two earmuffs attached to an elastic strap.

Furthermore, in accordance with some embodiments of the present invention, the device includes a pump and a selection lever that is adjustable such that the pump is operable to lengthen the earplug or to inflate the inflatable structures.

Furthermore, in accordance with some embodiments of the present invention, the selection lever is further adjustable to enable deflation of the inflatable structures or to shorten the earplug.

Furthermore, in accordance with some embodiments of the present invention, the plurality of inflatable structures includes three inflatable structures.

There is further provided, in accordance with some embodiments of the present invention, a method for sealing an ear against moisture, the method including: lengthening an earplug to a length that corresponds to a length of an ear canal; inserting the earplug into the ear canal; and laterally inflating an inflatable structure of the earplug to substantially fill a cross section of the ear canal and form a seal against penetration of moisture.

Furthermore, in accordance with some embodiments of the present invention, the method includes placing an earmuff over the ear such that a resilient rim of the earmuff forms a seal against penetration of moisture.

Furthermore, in accordance with some embodiments of the present invention, the method includes operating a pump to cause air within a space of the earmuff to flow through a hygroscopic material.

### BRIEF DESCRIPTION OF THE DRAWINGS

In order to better understand the present invention, and appreciate its practical applications, the following Figures are provided and referenced hereafter. It should be noted that the Figures are given as examples only and in no way limit the scope of the invention. Like components are denoted by like reference numerals.

FIG. 1 schematically illustrates an earplug of an ear protector, in accordance with an embodiment of the present invention.

FIG. 2 is a schematic cross section of an earplug of an ear protector, in accordance with an embodiment of the present invention.

FIG. 3A schematically illustrates an earplug, in accordance with an embodiment of the present invention, prior to elongation.

FIG. 3B schematically illustrates the earplug of FIG. 3A, after elongation and insertion into an ear canal.

FIG. 3C shows the elongated and inserted earplug of FIG. 3B after inflation of laterally inflatable structure.

FIG. 4 schematically shows an earmuff assembly, equipped with a rigid bow, of an ear protector device in accordance with an embodiment of the present invention.

FIG. 5 is a schematic cross section of an ear protection earmuff of an ear protector device configured for moisture removal, in accordance with an embodiment of the present invention.

FIG. 6 schematically shows an earmuff assembly, equipped with an elastic strap, of an ear protector device in accordance with an embodiment of the present invention.

FIG. 7 schematically shows an alternative design of an earplug with an inflation mechanism, in accordance with an embodiment of the present invention.

FIG. 8A schematically illustrates a configuration for lengthening the earplug shown in FIG. 7.

FIG. 8B schematically illustrates a configuration for laterally inflating the earplug shown in FIG. 7.

FIG. 8C schematically illustrates a configuration for deflating laterally inflated structures of the earplug shown in FIG. 7.

FIG. 8D schematically illustrates a configuration for shortening the earplug shown in FIG. 7.

FIG. 9 is a flowchart depicting a method for sealing an ear canal against penetration of moisture, in accordance with an embodiment of the present invention.

### DETAILED DESCRIPTION OF THE INVENTION

In the following detailed description, numerous specific details are set forth in order to provide a thorough understanding of the invention. However, it will be understood by those of ordinary skill in the art that the invention may be practiced without these specific details. In other instances, well-known methods, procedures, components, modules, units and/or circuits have not been described in detail so as not to obscure the invention.

Although embodiments of the invention are not limited in this regard, discussions utilizing terms such as, for example, “processing,” “computing,” “calculating,” “determining,” “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium (e.g., a memory) that may store instructions to perform operations and/or processes. Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. Unless explicitly stated, the method embodiments described herein are not constrained to a particular order or sequence. Additionally, some of the described method embodiments or elements thereof can occur or be performed simultaneously, at the same point in time, or concurrently. Unless otherwise indicated, us of the conjunction “or” as used herein is to be understood as inclusive (any or all of the stated options).

In accordance with an embodiment of the present invention, an ear protector is configured to protect the ear canal against entry or penetration of moisture. The ear protector includes an earplug that is configured to be inserted into the ear canal. The earplug may be disposable, intended for a single use. For example, the ear protector may include a fitting or connector to which a disposable earplug may be attached. Alternatively or in addition, the earplug may be configured to be cleaned, disinfected, or otherwise treated between uses to enable at least limited reuse (e.g., up to a maximum number of reuses) of the earplug.

The earplug includes a plurality of laterally inflatable structures that are arranged along a longitudinal axis of the earplug. For example, each inflatable structure is made of a flexible or elastic material (e.g., an elastomer). Thus, when the earplug is inserted into the ear canal, each of the inflatable structures is located at a different point along the ear canal. For example, each inflatable structure may be in the form of an inflatable bulb, ring, balloon, or similar shape. Prior to inflation of the inflatable structures, the diameter of the earplug is sufficiently small to enable convenient insertion into the ear canal. For example, the diameter of the earplug prior to inflation may be about 2 mm or another diameter. After inflation, the diameter of the inflatable structures may be about 12 mm or another diameter. In some cases, e.g., when intended for use in an ear on which a procedure such as mastoidectomy has been performed, the diameter of the inflatable structures after inflation may be about 18 mm, or another diameter. For example, different standard earplug diameters may be provided (e.g., labeled as intended for use by people with differently sized ear canals, of different ages, or having undergone various medical procedures).

Each of the laterally inflatable structures may be inflated by introduction of pressurized air or another gas. For example, a pump or other mechanism may be operable from outside of the ear to introduce pressurized as into the laterally inflatable structures. When the pressurized gas is introduced into the laterally inflatable structures, each laterally inflatable structure is inflated and expands. Each of the laterally inflatable structures may expand so as to fill a cross section of the ear canal at the longitudinal location of that laterally inflatable structure. When the cross section is filled, each point on a contiguous line on the outer perimeter of each of the laterally inflatable structures is in contact with a corresponding point on the wall of the ear canal.

The earplug may include an inflation system that includes an arrangement of tubes or pipes for conducting pressurized gas from a pressurized gas source to the laterally inflatable structures. A one-way valve or other mechanism may be provided to retain pressurized gas within the inflation system after inflation of the laterally inflatable structures. For example, the inflation system may include a tube that extends longitudinally within the earplug. The pressurized gas source is connectable to a proximal end of the longitudinal tube. One or more tubes may extend radially outward from the longitudinal tube to each of the inflatable structures. Thus, each radial tube may conduct the pressurized gas from the longitudinal tube to one of the laterally inflatable structures in order to inflate that structure.

The pressurized gas source may include a mechanically or electrically operated pump. The pump may be operated to pump air from the ambient atmosphere into the inflation system. The pump may be incorporated into a non-disposable component of the ear protector. A connector for connecting a disposable ear plug to the ear protector may include an appropriate fitting for connecting a tube of the inflation system to the pump. Alternatively or in addition, a pump may be incorporated into the earplug itself.

The pressurized gas source may include a canister, tank, or other container of a compressed gas. The pressurized gas container may be provided with a valve that enables controlling inflation of the inflatable structures. The pressurized gas container may be incorporated into a non-disposable component of the ear protector. Alternatively or in addition, pressurized gas container may be incorporated into the earplug itself (e.g., containing sufficient gas for a single use).

When thus inflated and expanded, each laterally inflatable structure that fills the local cross section of the ear canal forms a barrier to the passage of water or another liquid. For example, an outer surface of each laterally inflatable structure may be made of a material that is impermeable to the passage of water, such as an elastomer or other polymer. In addition, each laterally inflatable structure, when inflated, exerts a friction force with respect the wall of the ear canal. The friction force may resist sliding of the earplug relative to the ear canal, thus holding the earplug in place.

A separation distance between the laterally inflatable structures is adjustable. For example, the ear plug may include a longitudinally extendible element whose length may be adjusted. The inflatable structures are attached to the longitudinally extendible element in such a manner that separation between adjacent laterally inflatable structures is increased when the length of the longitudinally extendible element is increased.

The longitudinally extendible element may include an inflatable tube whose length increases when pressurized gas is introduced into the tube. For example, an inflatable tube may include longitudinally inflatable segments that are located between the laterally inflatable structures. The laterally inflatable structures may be placed at regions between the longitudinally inflatable segments (e.g., such that the laterally inflatable structures are not longitudinally stretched when the longitudinally extendible element is lengthened). A longitudinally inflatable segment may include a segment where a flexible tube material is accordion-folded or otherwise folded or collapsed to enable expansion when inflated with pressurized gas. A longitudinally inflatable segment may include a segment of elastic material that may linearly expand when inflated with pressurized gas.

Pressurized gas for longitudinally extending the inflatable tube may be provided via a system of one or more tubes. The system of tubes for longitudinally extending the inflatable tube may be separate from the inflation system for providing pressurized gas to inflate the laterally inflatable structures. For example, a separate pump or pressurized gas container may be provided to provide pressurized gas to longitudinally extend the inflatable tube. A mechanism to operate separate pumps may be configured to reduce or prevent inadvertent operation of the wrong pump. For example, one mechanically operated pump may be operated by a rotational motion, while the other is operated by a pushing motion.

The length of the separation distance may be adjusted to fit an ear canal of a particular person. In some cases, a length may be adjusted, prescribed, or recommended by a person who is trained in fitting an ear plug to an ear canal. Such a trained person may include a physician or other medical professional, or a trained technician. The length of the part that is inserted into the ear may be limited to a maximum length (e.g., 1.8 cm, or another length). The length may be adjusted so as to prevent a distal end of the ear plug (e.g., an end that is most deeply inserted into the ear canal) from contacting or approaching the eardrum. For example, different standard lengths of earplugs may be provided. A length that is suitable for insertion into a particular ear canal may be selected. For example, a standard lengths may be labeled as suitable for use in ears of people of different sizes (e.g., labeled as suitable for a particular age range, male or female, weight or height range, or other standard size range). An earplug may be selected that fits the person into whose ear the earplug is to be inserted (e.g., taking into account any deviations in size of a particular person from a typical population). In some cases, a user of the ear protector may be trained to correctly adjust the length by being sensitive to auditory or tactile sensations when adjusting the length while the earplug is inserted into the ear.

If a length is prescribed, the user may be provided with a tool or gauge to extend the length of the earplug to the prescribed length. For example, the user may be provided with a chamber or tube with a length that is adjusted to correspond to a recommended or prescribed length of the earplug. The user may place the earplug into the chamber and inflate until the length of the earplug fills the chamber. As another example, the user may be provided with a graduated or otherwise marked tube, chamber, ruler, caliper, or other device to measure a length of the earplug. The user may inflate the earplug until its length is long enough to provide protective effect.

Thus, the length of the earplug may be adjusted in accordance with the length of the ear canal, and the inflatable structures may be inflated such as each inflatable structure completely fills its corresponding local cross section of the ear canal. When so inflated, a series of separated barriers are formed. Thus, moisture that succeeds in passing (e.g., seeps around) an outer barrier may be prevented from further penetration into the ear canal by the inner barriers.

The mechanism for longitudinal extension of the earplug may be identical with, or may share components with, the mechanism for inflation of the laterally inflatable structures. For example, the mechanism may be provided with a control for directing a pressurized gas from a pressurized gas source either to longitudinally extend the earplug or to inflate the laterally inflatable structures. Similarly, the mechanism may be controllable to allow the escape of pressurized gas to deflate the laterally inflatable structures or to shorten the earplug.

In accordance with an embodiment of the present invention, the ear protector may include an earmuff. The earmuff may be placed over an ear into which the earplug is inserted in order to provide a further barrier to penetration of water. In accordance with an embodiment of the present invention, the earmuff may include a mechanism for providing a dry atmosphere in the space that is formed between the earmuff and the ear. For example, the mechanism may include a mechanism for removing moisture from the air that is trapped in the space between the earmuff and the ear and surrounding skin surface. Alternatively or in addition, the earmuff may include a source (e.g., canister) of dry air or gas that may be released into the space to displace moist air.

In accordance with an embodiment of the present invention, the earplug may be mounted to the earmuff. For example, the earplug may be made to extend inward from an inward-facing side of the earmuff.

An outer surface of the earmuff may be covered with a material that is impenetrable to water or other liquids. For example, the outer surface may be covered with or consist of a plastic or polymer, metal, or other impenetrable material.

A rim of the earmuff is configured to contact the side of the user's head surrounding the ear when worn on an ear. The rim may include a resilient material (e.g., rubber or another elastic or resilient polymer) that is configured to conform to the surface topography of the head surface surrounding the ear. Thus, the rim material may form a seal that is resistant to seepage of water into the interior of the cup of the earmuff from outside of the ear protector.

A mechanism may provide a force to hold the earmuff (typically both earmuffs concurrently) against the side of the head in order to form an effective seal. For example, a rigid headband in the form of bow-shaped tension bar may be adjustable to press the earmuffs against the side of the head. Such a rigid headband may provide a seal that is sufficient under conditions where the ambient liquid pressure is relatively low. Such conditions may be present when the head is not immersed in the liquid, for example when showering or when bathing in a shallow tub. As another example, an elastic headband in the form of an elastic strap may be configured to hold the earmuffs against the side of the head. Such an elastic headband may be adjustable to provide sufficient tension to provide an effective seal under conditions of moderate ambient liquid pressure (e.g., the head immersed in water up to a maximum depth, e.g., about 2 meters). Such conditions may occur when swimming.

An earmuff may be provided with a drying mechanism for removing moisture from air that is trapped between the earmuff and the user's head. For example, an interior of the earmuff may include a hygroscopic material to act as a desiccant to absorb moisture from the air. The hygroscopic material may be held in a desiccant chamber that is separated by an impermeable barrier from the remainder of the interior of the earmuff. A pumping mechanism may pump air from the interior of the earmuff via one or more inlet vents into the desiccant chamber. The pumped air may be forced through the hygroscopic material where moisture may be removed from the air. The air is forced out of the desiccant chamber and back into the interior of via an outlet vent. The air that exits from the desiccant chamber via the outlet vent may be dried, with at least some the moisture removed.

Thus, an ear protector, in accordance with an embodiment of the present invention, may offer multiple layers of protection for preventing moisture from reaching the vicinity of the eardrum. The impermeable outer surface of the earmuff and the seal between the earmuff and the side of the user's head may restrict the amount of moisture that enters the space between the earmuff and the user's ear. The drying mechanism may remove moisture that penetrates into the space between the earmuff and the user's ear. Finally, the inflatable structures of the earplug form a series of barriers to prevent penetration of moisture into the ear canal and to the eardrum.

An ear protector, in accordance with an embodiment of the present invention, may be used to protect an ear from excessively loud noises.

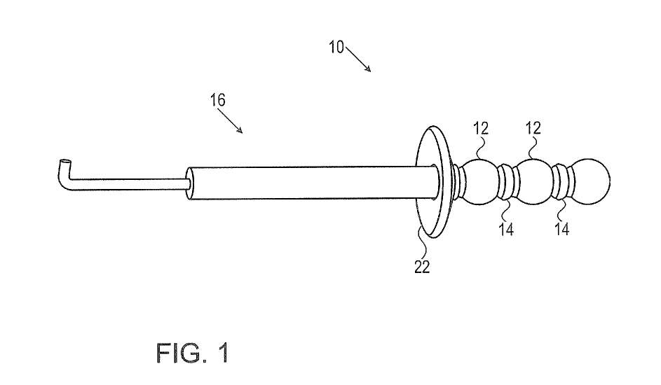

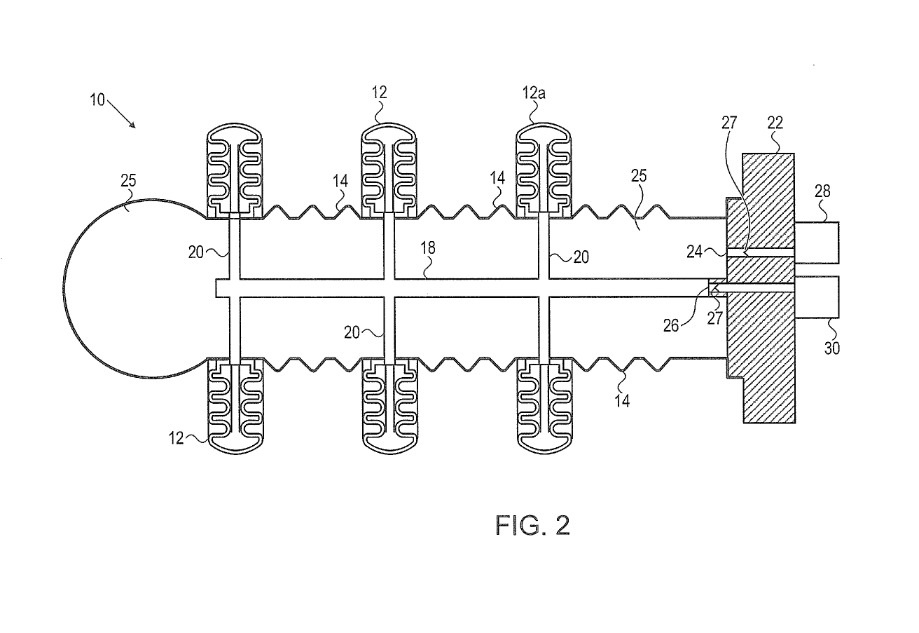

FIG. 1 schematically illustrates an earplug of an ear protector, in accordance with an embodiment of the present invention. FIG. 2 is a schematic cross section of an earplug of an ear protector, in accordance with an embodiment of the present invention.

Earplug 10 is configured to be inserted into an ear canal. Base 22 is wider than more distal (relative to an observer outside of the ear) portions of earplug 10 (e.g., prior to inflation of laterally inflatable structures 12). Base 22 of earplug 10 may be sufficiently wide to prevent its insertion into an ear canal. Thus, earplug 10 may be removed from an ear canal after insertion via structure (e.g., a handle, tab, loop, string, wire, or other structure) that is attached to an outwardly (from the ear) facing side of base 22. Alternatively or in addiction, base 22 may include structure that is configured to attach to an earmuff or other component of an ear protector. Base 22 includes structure for connection to one or more pressurized gas sources, such as to elongation gas source 28 or to lateral inflation gas source 30. For example, base 22 may include elongation gas duct 24, lateral inflation gas duct 26, or both.

For example, base 22 may be configured to attach to an earplug fitting 16. Earplug fitting 16 may be incorporated into an earmuff of an ear protector, or may be insertable into an appropriate channel of an earmuff. Earplug fitting 16 may include one or more tubes or conduits for connecting elongation gas duct 24 or lateral inflation gas duct 26 to a corresponding elongation gas source 28 or lateral inflation gas source 30.

Earplug 10 includes a plurality of laterally inflatable structures 12 separated from one another, or from base 22, by longitudinally inflatable segments 14. For example, an earplug 10 may include three laterally inflatable structures 12 and three longitudinally inflatable segments 14, as shown. An earplug may include another number of (e.g., two, or more than three of) laterally inflatable structures 12, of longitudinally inflatable segments 14, or both.

Elongation of a longitudinally inflatable segment 14 between two laterally inflatable structures 12 increases the separation distance between those two laterally inflatable structures 12. Similarly, elongation of a longitudinally inflatable segment 14 between base 22 and a first laterally inflatable structure 12a increases a distance between the base 22 and first laterally inflatable structure 12a.

For example, each longitudinally inflatable segment 14 may include a segment of a flexible material (e.g., a polymer) that is impermeable to the inflating gas. The material may be longitudinally accordion folded as shown. Thus, when pressurized gas is introduced into interior gas channel 25 of earplug 10, the increased internal gas pressure may cause the accordion folds of each longitudinally inflatable segment 14 to straighten. Straightening the accordion folds thus results in an increase in the length of each longitudinally inflatable segment 14.

As another example, each longitudinally inflatable segment 14 may be constructed of an anisotropic elastic material (e.g., an elastomer) that is impermeable to the inflating gas. For example, an elastic material of longitudinally inflatable segment 14 may include constraining structure. The constraining structure may constrain longitudinally inflatable segment 14 so as to restrict stretching primarily to the longitudinal direction. For example, an elastic material of which longitudinally inflatable segment 14 is constructed may include constraining structure in the form of circular inelastic fibers arranged around the axial direction to inhibit lateral stretching.

Each longitudinally inflatable segment 14 may be configured so as not to extend beyond a maximum longitudinal extent. For example, the quantity of folded material may be limited such that the length of earplug 10 when all of the folds are straightened does not exceed a maximum length. An elastic material may be constructed (e.g., with longitudinally oriented inelastic fibers) so as to limit the extent to which each longitudinally inflatable segment 14 may be stretched.

Elongation gas source 28 is configured to introduce a pressurized gas into interior gas channel 25 via elongation gas duct 24. For example, elongation gas source 28 may include a pump for pumping air from the ambient atmosphere into interior gas channel 25. The pump may be manually operated. For example, the pump may include a handle that may be manually operated to pump air into interior gas channel 25. The handle may be a rotary handle, configured to be rotated (e.g., turned or rocked back and forth) about an axis. The handle may be configured to be moved (e.g., pushed or slid) linearly back and forth along an axis. The pump may be motorized, provided with an electric power source and switch or other control to control longitudinal inflation of longitudinally inflatable segments 14. As another example, elongation gas source may include a canister, tank, bottle, can, or other container containing pressurized gas. The pressurized gas may include air or another gas. A valve (e.g., on elongation gas source 28 or on elongation gas duct 24) may regulate flow of pressurized gas from elongation gas source 28 to interior gas channel 25.

Elongation gas duct 24 may be provided with a valve 27 to prevent escape of gas from interior gas channel 25 after elongation gas source 28 ceases to provide gas to interior gas channel 25. For example, valve 27 may include a one-way valve. Valve 27 may include structure to enable deliberate release of pressurized gas from interior gas channel 25 (e.g., when over-inflated or when deflating after use).

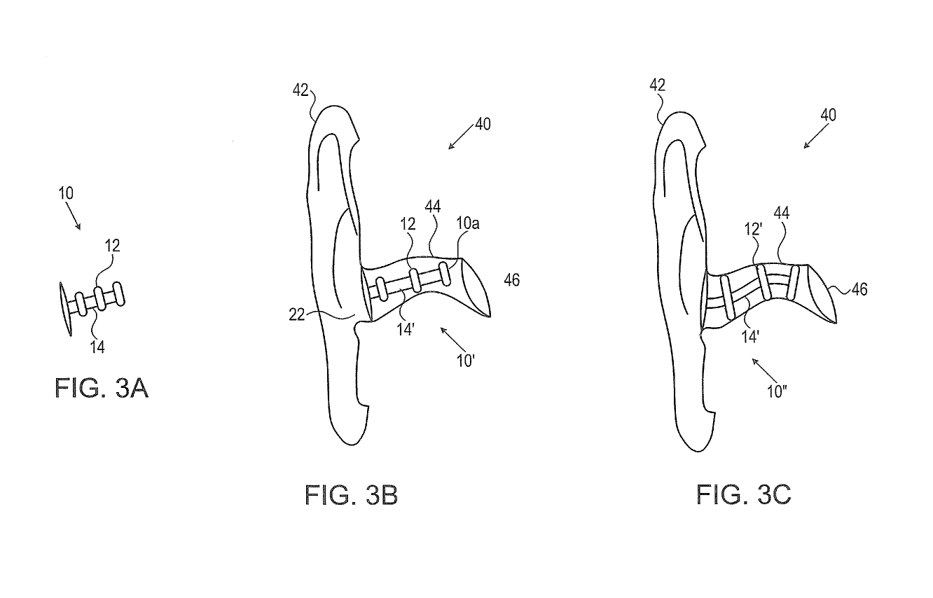

A length of earplug 10 may be expanded to a target length prior to insertion into an ear canal. The target length may be less than the distance of eardrum 46 from base 22 when earplug 10 is fully inserted into ear canal 44 (see FIG. 3B). For example, the target length may be determined or estimated by examination of ear canal 44 by a trained person using an otoscope or other instrument. A length of ear canal 44 may be determined by an instrument that is designed or configured to measure a length of ear canal 44 up to eardrum 46 (e.g., using acoustical or optical distance measurement techniques). The target length of earplug 10 may be determined as sufficiently less than the length of the ear canal so as to ensure that earplug 10 does not physically contact eardrum 46 under foreseeable sets of circumstances.

Once a target length is determined, (e.g., from a selection of earplug target lengths, e.g., in accordance with the age or size of the user), a user of earplug 10 may operate or use elongation gas source 28 to expand earplug 10 to the target length. For example, various earplugs 10 may be produced or manufactured in different sizes, each size being characterized by a maximum length to which earplug 10 may be expanded. Once a target length is determined, a size of earplug 10 may be prescribed, selected, or customized with a maximum size that is equal to or less than the determined target length. As another example, a user may be provided with, obtain, or utilize a suitable structure for determining when the length of earplug 10 has been expanded to the target length. For example, the user may be provided with a fixture that includes a cavity whose length is substantially equal to the target length. The user may then operate elongation gas source 28 to expand the length of earplug 10 to fill the cavity. A fixture may be provided that automatically measures the length of earplug 10 (e.g., using optical or acoustic techniques) and automatically operates an electronically controlled pump of elongation gas source 28 or a valve to stop inflation of interior gas channel 25 when the target length is reached. Other length measuring instruments, such as rulers, calipers, ruled tubes, or other manual or electronic length measuring devices, tools, or instruments may be used to assist in expanding the length of earplug 10 to the target length.

Each laterally inflatable structure 12 is arranged in an axially symmetric manner around earplug 10. For example, each laterally inflatable structure 12 may have a ring-like, toroidal, or knoblike form.

When each laterally inflatable structure 12 is filled with pressurized gas, it expands laterally (e.g., radially) outward from earplug 10. Typically, lateral expansion of each laterally inflatable structure 12 when inserted into an ear canal 44 (FIG. 3B) is limited by the walls of ear canal 44 that surround that laterally inflatable structure 12.

Lateral inflation gas source 30 may be operated to inflate laterally inflatable structures 12. For example, lateral inflation gas source 30 may include a pump for pumping air from the ambient atmosphere into main lateral inflation channel 18. Main lateral inflation channel 18 is arranged longitudinally within earplug 10. The pump may be manually operated. For example, the pump may include a handle that may be manually operated to pump air into main lateral inflation channel 18. The handle may be a rotary handle, configured to be rotated (e.g., turned or rocked back and forth) about an axis. The handle may be configured to be moved (e.g., pushed or slid) linearly back and forth along an axis. The handle may be configured to operate differently from a handle of a pump of elongation gas source 28, so as to avoid unintentional operation of the wrong pump. The pump may be motorized, provided with an electric power source and switch or other control to control operation of the pump to laterally inflate laterally inflatable structures 12. As another example, elongation gas source may include a canister, tank, bottle, can, or other container containing pressurized gas. The pressurized gas may include air or another gas. A valve (e.g., on lateral inflation gas source 30 or on lateral inflation gas duct 26) may regulate flow of pressurized gas from lateral inflation gas source 30 to main lateral inflation channel 18.

One or more radial lateral inflation channels 20 connect a common main lateral inflation channel 18 with each laterally inflatable structure 12. Thus, pressurized gas from lateral inflation gas source 30 may be conducted to each laterally inflatable structure 12 to inflate each laterally inflatable structure 12.

Lateral inflation gas duct 26 may be provided with a valve 27 to prevent escape of gas from main lateral inflation channel 18 after lateral inflation gas source 30 ceases to provide gas to main lateral inflation channel 18. For example, valve 27 may include a one-way valve. Valve 27 may include structure to enable deliberate release of pressurized gas from main lateral inflation channel 18. For example, gas may be released to partially or fully deflate laterally inflatable structures 12 to enable removal of earplug 10 from the ear canal after use.

Earplug 10 may be used to seal an ear canal to prevent moisture from reaching the eardrum. A method to seal an ear canal includes elongating earplug 10 to a desired length, inserting the elongated earplug into the ear canal, and inflating laterally inflatable structures 12 to each form a seal against penetration of moisture into the ear canal to the ear drum. Various stages of the method are shown in FIGS. 3A-3C.

FIG. 3A schematically illustrates an earplug, in accordance with an embodiment of the present invention, prior to elongation. FIG. 3B schematically illustrates the earplug of FIG. 3A, after elongation and insertion into an ear canal.

Earplug 10 of FIG. 3A has been elongated by longitudinal inflation of longitudinally inflatable segments 14 to form elongated segments 14′. Elongated earplug 10′ has been inserted into ear canal 44 of ear 40. The diameter (e.g., about 2 mm) or other lateral dimension of elongated earplug 10′ is sufficiently less than that of ear canal 44 to enable easy insertion of elongated earplug 10′ into ear canal 44. For example, elongated earplug 10′ may be inserted into ear canal 44 until further insertion is prevented by contact of base 22 with structure of outer ear 42. When so inserted, distal end 10a of elongated earplug 10′ does not contact eardrum 46.

After insertion of elongated earplug into ear canal 44, each laterally inflatable structure 12 may be inflated.

FIG. 3C shows the elongated and inserted earplug of FIG. 3B after inflation of laterally inflatable structure.

Each laterally inflatable structure 12 has been inflated to form an inflated sealing structure 12′. Each inflated sealing structure 12′ has expanded to fill the local cross section of ear canal 44. Thus, inflated sealing structures 12′ form a series of barriers to penetration of liquids or moisture into ear canal 44 to eardrum 46. Furthermore, contact between each inflated sealing structure 12′ and ear canal 44 resists (e.g., via friction or other mechanical forces) unintentional or accidental removal of inflated earplug 10″ from ear canal 44.

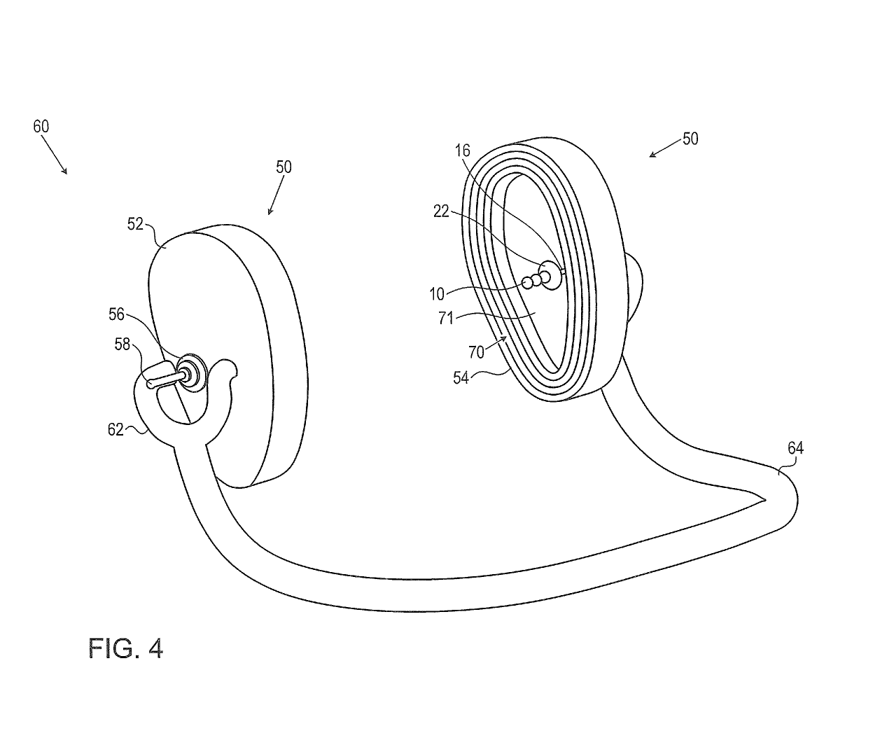

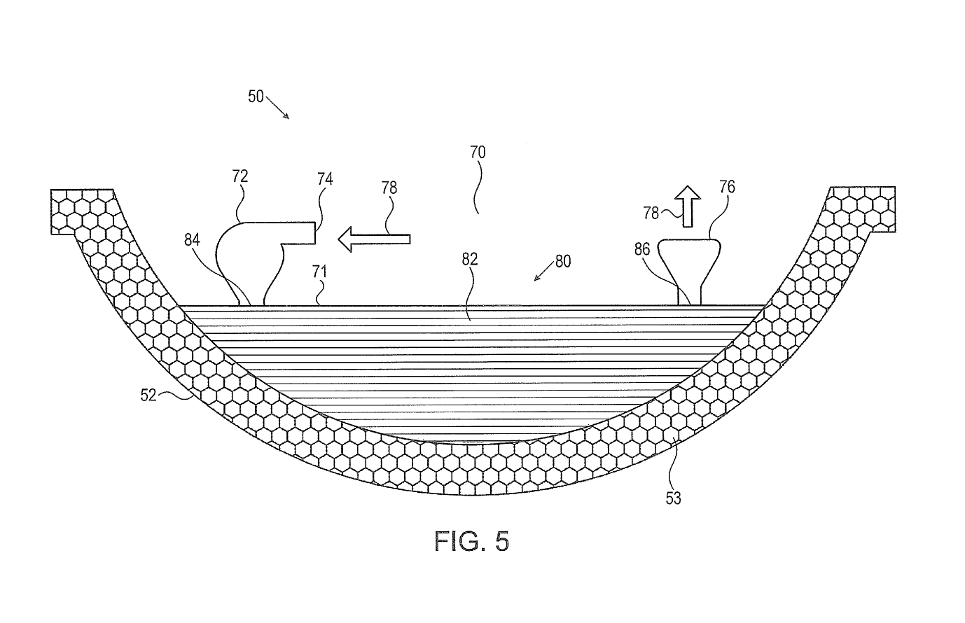

FIG. 4 schematically shows an earmuff assembly, equipped with a rigid bow, of an ear protector device, in accordance with an embodiment of the present invention.

Earmuff assembly 60 includes two protection earmuffs 50 connected by rigid bow 64. Rigid bow 64 attaches to each protection earmuff 50 via a bracket 62. For example, each protection earmuff 50 may be placed over one of the ears of a user. When so placed, rigid bow 64 may be positioned over the nape of the neck of the user. Rigid bow 64 may be adjusted (e.g., by moving one or more telescoping or sliding segments relative to one another) to provide a tension force. The provided tension may be sufficient to hold protector earmuffs 50 over the ears in the absence of strong external forces. For example, rigid bow 64 may provide sufficient tension force when the user is showering or is bathing in a shallow bathtub. Furthermore, rigid bow 64 may enable access to the user's nape and to the back of the user's head when washing those parts.

Each protection earmuff 50 is shaped to fit over an ear of the user. Typically, each protection earmuff 50 may be placed over the ear after insertion of an earplug 10 into the ear canal of the ear. In this case, there is no need for a physical connection of any kind between earplug 10 and protection earmuff 50.

In some cases, and as shown, an earplug 10 may be attachable to protection earmuff 50. For example, each protection earmuff 50 may be provided with a earplug fitting 16. Earplug fitting 16 may be permanently attached to protection earmuff 50, or may be attachable to protection earmuff 50 prior to, or together with, attachment of an earplug 10 to protection earmuff 50. An earplug 10 may be configured to connect to earplug fitting 16. Protection earmuff 50 may be shaped such that when placed over the user's ear, earplug 10 is inserted into the ear canal of the ear. A length of earplug 10 may be adjusted prior to attachment to earplug fitting 16 or to protection earmuff 50, or after attachment.

Earplug fitting 16 may include one or more conduits for connecting earplug 10 to one or sources of pressurized gas. For example, a source of pressurized gas for inflating laterally inflatable structures of earplug 10 may include inflation pump 56. Inflation pump 56 may be a manual pump operated by manipulation of pump handle 58. Alternatively or in addition, inflation pump 56 may be electrically (e.g., battery) operated, with pump handle 58 representing an electrical switch. When operated, inflation pump 56 may pump air via a conduit in earplug fitting 16 to inflate the laterally inflatable structures. In accordance with some examples, a source of pressurized gas may be provided on protection earmuff 50 for elongating earplug 10. For example, a single pump handle 58 may be operated with different motions (e.g., rotation, rocking, or pushing) in order to operate one or the other pumping process. In accordance with other examples, different handles may be provided for different pumps or pumping processes. In accordance with other examples, an earplug 10 may be elongated by a separate (e.g., detached) source of pressurized gas prior to attachment of earplug 10 to earplug fitting 16 or to protective earmuff 50.

Outer shell 52 of protection earmuff 50 is made of a material that is impermeable to moisture. For example, outer shell 52 may be constructed of a nonporous hard polymer, or out of another suitable material (e.g., glass or metal). In some cases, a nonporous hard outer shell 52 may enclose a less dense (e.g., lightweight) rigid inner shell 53 (e.g., as schematically shown in the cross section that is visible in FIG. 5). For example, inner shell 53 may include a rigid polymeric foam, or similar lightweight material.

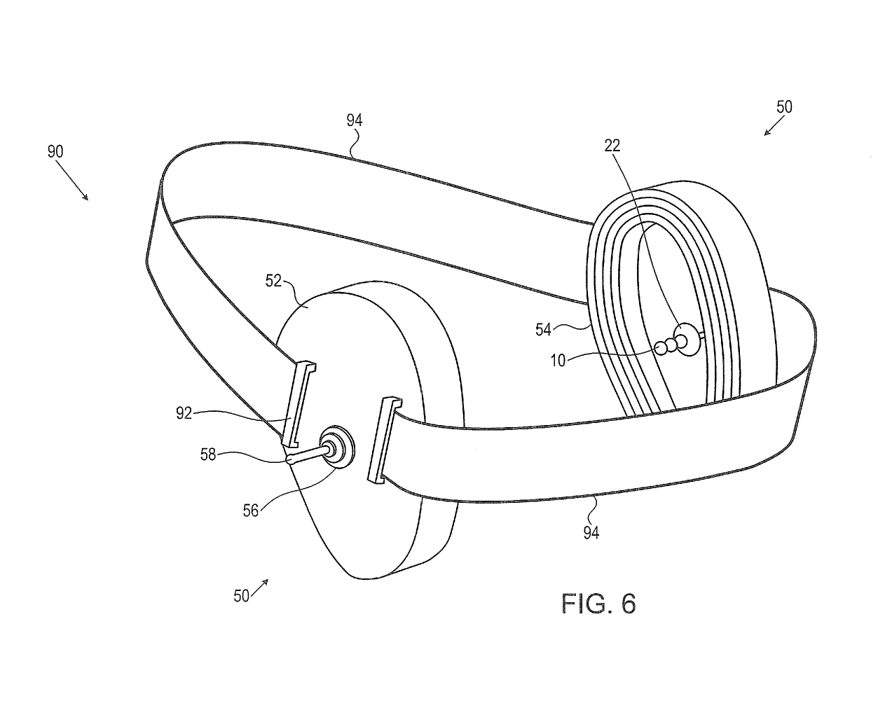

The edges of outer shell 52 may terminate in resilient rim 54. Resilient rim 54 may be constructed of a resilient material that is impermeable to moisture or liquids. For example, resilient rim 54 may be constructed of a nonporous elastomer. As another example, resilient rim 54 may include a porous elastomer that is enclosed within a layer of an impermeable flexible or elastic material. When protection earmuff 50 is held against the side of the user's head and over the user's ear, e.g., by rigid bow 64 or by elastic strap 64 (FIG. 6), resilient rim 54 may conform to the shape of the user's head. When conforming to the shape of the user's head, contact between resilient rim 54 and the user's head may form a seal that is impermeable to, or is resistant to seepage by, water or another liquid.

When protection earmuff 50 is held to a user's head over the user's ear, a pocket of air is typically trapped in space 70 between interior wall 71 of protection earmuff 50 and the side of the head. Moisture may have been trapped within space 70, or moisture may seep into space 70 (e.g., by a temporary opening in the seal formed by resilient rim 54). Protection earmuff 50 may be configured to remove moisture from air trapped within space 70.

FIG. 5 is a schematic cross section of an ear protection earmuff of an ear protector device configured for moisture removal, in accordance with an embodiment of the present invention.

Protection earmuff 50 includes moisture removal cassette 80. Moisture removal cassette 80 includes hygroscopic material 82. In some cases, moisture removal cassette 80 may be removable and replaceable. For example, interior wall 71 of protection earmuff 50 may be configured to open to enable insertion or removal of a moisture removal cassette 80. As another example, interior wall 71 may be a surface of moisture removal cassette 80. In this example, interior surfaces of protection earmuff 50 may include structure for holding moisture removal cassette in place, or to enable attachment of moisture removal cassette 80. In some cases, moisture removal cassette 80 may be permanently attached to protection earmuff 50.

Air from inside space 70 may enter moisture removal cassette 80 via one or more intake openings 84. Alternatively or in addition, intake opening 84 may represent a boundary (e.g., interior wall 71) that is permeable to the passage of air, or that includes patches or regions that are permeable to the passage of air. Air may exit from moisture removal cassette 80 to space 70 via one or more outflow openings 86. Alternatively or in addition, outflow opening 86 may represent a boundary (e.g., interior wall 71) that is permeable to the passage of air, or that includes patches or regions that are permeable to the passage of air.

Intake opening 84 may be provided with a pump 72 for pumping air from space 70 and pump intake 74 through intake opening 84. For example, pump 72 may include a battery-operated electric motor for operation of pump 72. Pump 72 may generate airflow 78 from space 70 via pump intake 74 and intake opening 84 through hygroscopic material 82, and out through outflow opening 86 back into space 70. Outflow opening 86 may be provided with outflow structure 76. For example, outflow structure 76 may represent a vent that directs outflowing air to a desired region of space 70, or to a plurality of regions within space 70.

For example, pump 72 may be turned on prior to placing protection earmuff 50 on a user's ear. Pump 72 may be turned on using an externally located switch after placement of protection earmuff 50 on the user's. Pump 72 may be configured to operate automatically whenever protection earmuff 50 is placed on a user's ear. For example, structure within protection earmuff 50 may be provided with a contact or proximity switch that is operated upon contact of protection earmuff 50 with a user's head. For example, a pressure or contract switch may be placed within resilient rim 54 (FIG. 4).

Moist air that passes through hygroscopic material 82 may be dried. For example, moisture in the air may adhere to, or react chemically with, hygroscopic material 82. Hygroscopic material 82 or moisture removal cassette 80 may be provided with an indicator that indicates if hygroscopic material 82 has become saturated with moisture. When saturated, moisture removal cassette 80 may be replaced, or processed (e.g., dried or heated) to remove the moisture. For example, the indicator may include a material that changes color when in accordance with the moisture content of hygroscopic material 82. The color may be viewed via a transparent section of moisture removal cassette 80 or of interior wall 71, or otherwise. An indicator may be provide on an exterior surface of protection earmuff 50, e.g., on outer shell 52, to enable or facilitate timely detection of saturation of hygroscopic material 82.

Alternatively or in addition to passage of air through hygroscopic material 82, protection earmuff 50 may include a canister or other source of a dry gas such as dry air. The dry gas may be caused to flow through space 70 or hygroscopic material 82 to maintain a low level of humidity in space 70.

A protection earmuff 50 may be used in an environment in which shear forces may be present. For example, a shear force may include a flow of water relative to protection earmuff 50. Such a flow of water may result, for example, when a user is swimming through water or is standing in the presence of waves. Such a shear force may tend to move or remove protection earmuff 50. Failure to prevent such movement could result in entry of water into the ear canal, or could cause discomfort or pain. An ear protector device may be configured to prevent movement of protection earmuff 50 when used in the presence of such shear forces.

FIG. 6 schematically shows an earmuff assembly, equipped with an elastic strap, of an ear protector device in accordance with an embodiment of the present invention.

Strap earmuff assembly 90 includes two protection earmuffs 50 connected by elastic straps 94. Elastic straps 94 attach to each protection earmuff 50 via strap brackets 92. For example, each protection earmuff 50 may be placed over one of the ears of a user. When so placed, elastic straps 94 may be tightened around the user's head. The tension force that is provided by elastic straps 94 may hold protector earmuffs 50 over the ears in the presence of shear forces. Thus, strap earmuff assembly 90 may be used by a user who is swimming, standing in waves, or participating in typical activities at a beach or swimming pool.

In accordance with an embodiment of the present invention, an earplug of the ear protection device may be provided with an inflation mechanism. The inflation mechanism may be operated to perform one or more operations including adjusting a length of the earplug or adjusting lateral inflation of laterally inflatable structures of the earplug.

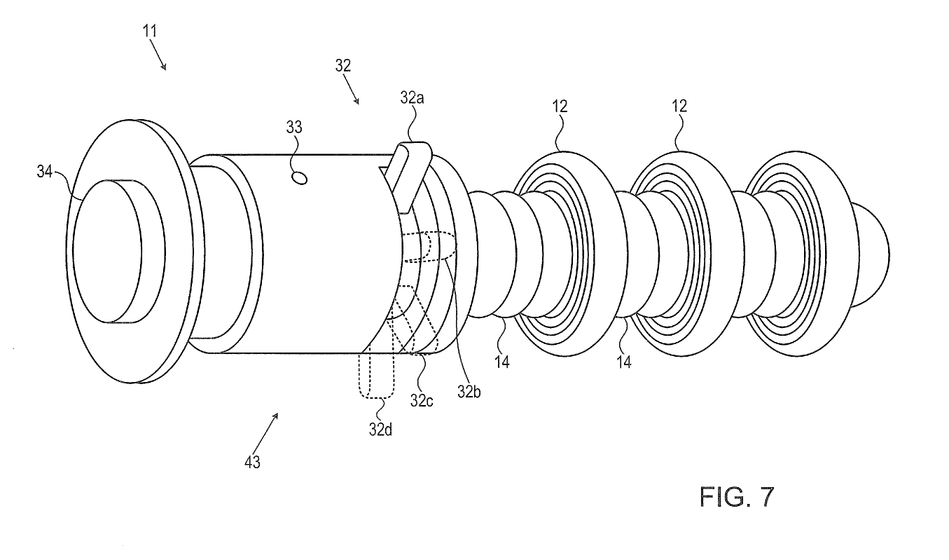

FIG. 7 schematically shows an alternative design of an earplug with an inflation mechanism, in accordance with an embodiment of the present invention.

Earplug 11 includes inflation assembly 43. Inflation assembly 43 includes selection lever 32 and pump plunger 34. Selection lever 32 may be moved to one or more positions (e.g., one of four positions 32a-32d). A mechanism of operation of selection lever 32 may include stops or a locking mechanism to prevent or inhibit accidental or unintentional movement of selection lever 32 from one of lever positions 32a-32d to another.

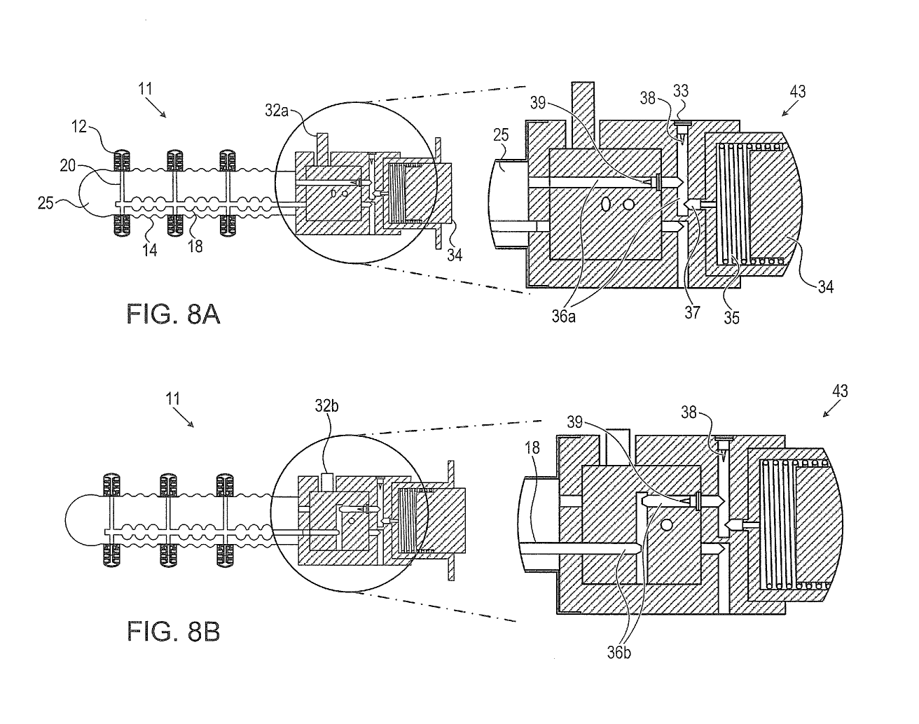

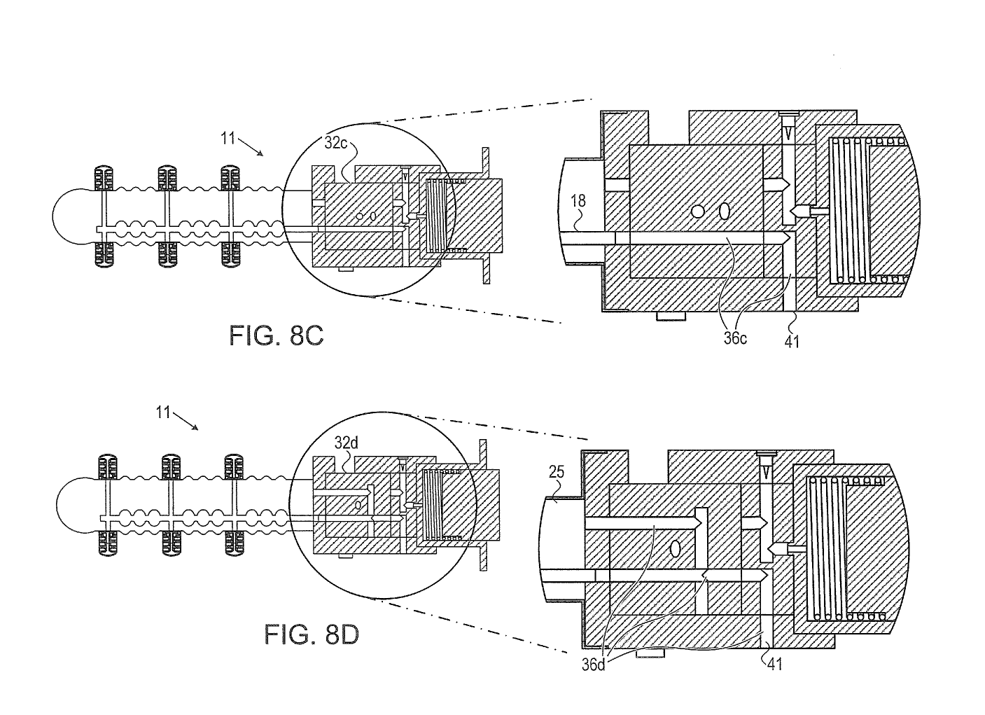

With some positions of selection lever 32, pressing pump plunger 34 inward may force air into earplug 11. Forcing air into earplug 11 may lengthen longitudinally inflatable segments 14 (e.g., at lever position 32a) or laterally inflate laterally inflatable structures 12 (e.g., at lever position 32b). In some positions of selection lever 32, air may be released from earplug 11 (e.g., via outflow hole 41 in FIG. 8C). For example, releasing air from earplug 11 may enable deflation of laterally inflatable structures 12 (e.g., at lever position 32c) or may enable shortening of longitudinally inflatable segments 14 (e.g., at lever position 32d). The lever positions may be otherwise associated with the functions, or with other functions.

FIG. 8A schematically illustrates a configuration for lengthening the earplug shown in FIG. 7.

At lever position 32a, pushing pump plunger 34 inward (e.g., using a finger) forces air out of pump passage 37, via air passage configuration 36a and one-way valve 39, into interior gas channel 25. Forcing air into interior gas channel 25 may inflate longitudinally inflatable segments 14, thus lengthening earplug 11. Upon release of pump plunger 34, spring 35 pushes pump plunger 34 outward. When pump plunger 34 is pushed outward, air may be drawn into inflation assembly 43 via intake hole 33 and one-way valve 38. Further pushing of pump plunger 34 may further inflate longitudinally inflatable segments 14. Longitudinal inflation of longitudinally inflatable segments 14 may continue until the length of earplug 11 is suitable for insertion into an ear canal.

FIG. 8B schematically illustrates a configuration for laterally inflating the earplug shown in FIG. 7.

At lever position 32b, pushing pump plunger 34 inward may force air out of pump passage 37, via air passage configuration 36b, one-way valve 39, and main lateral inflation channel 18, and into radial lateral inflation channels 20. Forcing air into radial lateral inflation channels 20 may inflate laterally inflatable structures 12. Inflation of laterally inflatable structures 12 may continue until earplug 11 has sealed an ear canal into which earplug 11 was inserted.

FIG. 8C schematically illustrates a configuration for deflating laterally inflated structures of the earplug shown in FIG. 7.

At lever position 32c, deflation (due to elastic restoring forces) of laterally inflatable structures 12 is enabled to force air out of laterally inflatable structures 12 via radial lateral inflation channels 20, main lateral inflation channel 18, and air passage configuration 36c out through outflow hole 41. Deflation of inflatable structures 12 may enable removal of earplug 11 from has sealed an ear canal into which earplug 11 was inserted.

FIG. 8D schematically illustrates a configuration for shortening the earplug shown in FIG. 7.

At lever position 32d, contraction (due to elastic restoring forces) of longitudinally inflatable segments 14 is enabled to force air out of interior gas channel 25 via air passage configuration 36d and out through outflow hole 41. Deflation of longitudinally inflatable segments 14 may shorten earplug 11, e.g., for convenient storage.

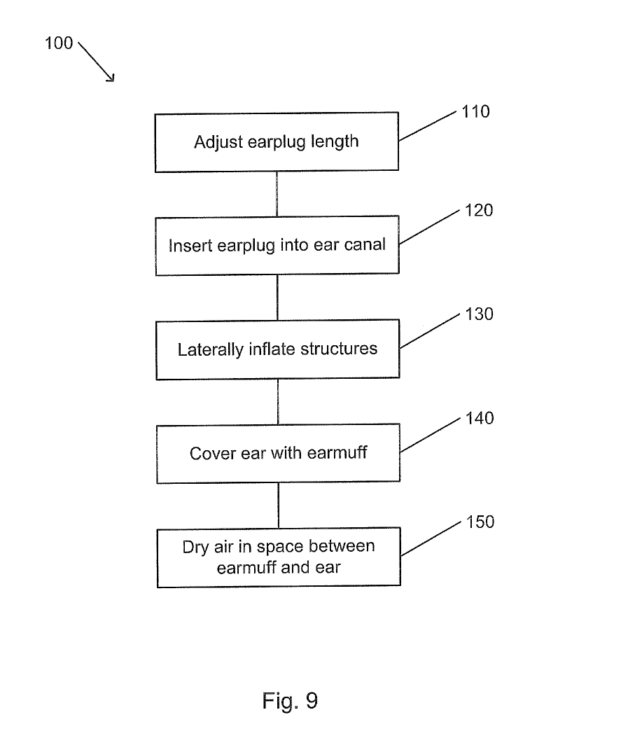

FIG. 9 is a flowchart depicting a method for sealing an ear canal against penetration of moisture, in accordance with an embodiment of the present invention.

It should be understood with respect to any flowchart referenced herein that the division of the illustrated method into discrete operations represented by blocks of the flowchart has been selected for convenience and clarity only. Alternative division of the illustrated method into discrete operations is possible with equivalent results. Such alternative division of the illustrated method into discrete operations should be understood as representing other embodiments of the illustrated method.

Similarly, it should be understood that, unless indicated otherwise, the illustrated order of execution of the operations represented by blocks of any flowchart referenced herein has been selected for convenience and clarity only. Operations of the illustrated method may be executed in an alternative order, or concurrently, with equivalent results. Such reordering of operations of the illustrated method should be understood as representing other embodiments of the illustrated method.

The operations of ear sealing method 100 may be performed prior to exposure of a user to water, moisture, or other liquids. For example, the user's ear may be affected by a condition that may be aggravated or may cause pain when water reaches the user's eardrum.

A length of an earplug of an ear protector device may be adjusted to a desired length (block 110). The desired length may correspond to a length of the user's ear canal such that the earplug does not contact the eardrum when fully inserted. A pump may be operated to longitudinally inflate the earplug to the desired length. Lengthening the earplug concurrently increases a distance between any two laterally inflatable structures of the earplug.

An earplug may be inserted into each ear canal (block 120).

When the earplug is fully inserted into the ear canal, laterally inflatable structures of the earplug may be inflated (block 130). Each inflatable structure, when inflated, forms a seal against penetration of water within the ear canal.

An earmuff of the ear protector device may be placed to cover each outer ear of the user (block 140). The earmuff may be tightened to form a seal that blocks water from reaching the outer ear.

Air that is trapped in a space between the earmuff and the user's ear may be dried (block 150). For example, the trapped air may be caused to flow through a moisture removal cartridge. For example, a pump may operate (e.g., concurrently with wearing the earmuff) to cause the flow. Flow of moist air through the cartridge may dry the air, e.g., to remove any moisture that may have seeped through the seal between the earmuff and the user's head.

1. An ear protector device comprising an earplug to insert into an ear canal of a user, the earplug including a plurality of inflatable structures arranged along a longitudinal axis of the earplug, a length of the earplug being extendible to increase a distance between two of the inflatable structures, each inflatable element being laterally inflatable when inserted into the ear canal so as to substantially fill a cross section of the ear canal.

2. The device of claim 1, comprising a longitudinally inflatable channel that is inflatable to lengthen the earplug.

3. The device of claim 2, further comprising a pump for inflating the channel.

4. The device of claim 1, wherein each structure of the plurality of inflatable structures is arranged axially symmetrically about the earplug.

5. The device of claim 1, wherein each structure of the plurality of inflatable structures is connected to a common longitudinal channel by a radial channel, the structure being inflatable via that radial channel.

6. The device of claim 1, comprising a widened base.

7. The device of claim 1, further comprising a pump for inflation of the inflatable structures.

8. The device of claim 1, further comprising at least one earmuff for covering an outer ear of the user.

9. The device of claim 8, wherein an outer surface of the earmuff is impermeable to moisture.

10. The device of claim 8, wherein the earmuff comprises a resilient rim for placement against a head of the user to form a seal against moisture.

11. The device of claim 8, wherein the earmuff comprises a cartridge containing a hygroscopic material.

12. The device of claim 11, comprising a pump to cause air from within the earmuff to flow through the hygroscopic material.

13. The device of claim 8, wherein said at least one earmuff comprises two earmuffs attached to a rigid bow.

14. The device of claim 8, wherein said at least one earmuff comprises two earmuffs attached to an elastic strap.

15. The device of claim 1, comprising a pump and a selection lever that is adjustable such that the pump is operable to lengthen the earplug or to inflate the inflatable structures.

16. The device of claim 15, wherein the selection lever is further adjustable to enable deflation of the inflatable structures or to shorten the earplug.

17. The device of claim 1, wherein said plurality of inflatable structures comprises three inflatable structures.

18. A method for sealing an ear against moisture, the method comprising:
lengthening an earplug to a length that corresponds to a length of an ear canal;
inserting the earplug into the ear canal; and
laterally inflating an inflatable structure of the earplug to substantially fill a cross section of the ear canal and form a seal against penetration of moisture.

19. The method of claim 18, further comprising placing an earmuff over the ear such that a resilient rim of the earmuff forms a seal against penetration of moisture.

20. The method of claim 19, further comprising operating a pump to cause air within a space of the earmuff to flow through a hygroscopic material.
